(19) United States
(12) Patent Application Publication
(10) Pub. No.: US 2004/0185067 A1
(43) Pub. Date: Sep. 23, 2004

(54) Material delivery method by skin absorption, peeling agent used for material delivery by skin absorption, peeling device, and disposable pad for peeling device

(76) Inventor: Machiko Daikuzono, Tokyo (JP)

Correspondence Address: MCDERMOTT WILL & EMERY LLP, 600 13TH STREET, N.W., WASHINGTON, DC, 20005-3096, US

(21) Appl. No.: 10/390,186

(22) Filed: Mar. 18, 2003

(51) Int. Cl. A61K 7/00

(52) U.S. Cl. 424/401; 601/2

(57) ABSTRACT

The present invention relates to a technique capable of carrying out peeling a barrier on the skin surface at a low cost and with ease, and moreover, uniformly and accurately without accompanying any pain. The delivery method of a substance via skin absorption of the present invention uses abrasive particles having an average particle diameter smaller than 1000 nm for peeling the skin surface, while a delivery substance is applied to the skin surface and the delivery substance is delivered from the peeled skin surface into the inside of the skin via skin absorption.

### BACKGROUND OF THE INVENTION

The present invention relates to a technique for delivery of medical substances, cosmetic substances and the like via skin absorption as well as a preferable peeling device therefor.

### FIELD OF THE INVENTION

When a medical substance, a cosmetic substance or the like is provided to a skin surface of a living body for delivering the substance inside the tissues of the living body via absorption from the skin surface, the existence of a barrier on the skin surface such as waste product, aging keratin and the like of the skin has been known as a factor to inhibit its absorption. Therefore, methods of peeling (exfoliation) the barrier on the skin surface prior to providing a delivery substance to the skin surface have been known.

As the peeling methods, tape peeling, a method using a medicine such as hyaluronic acid and the like, abrasion in which high-speed particles are sprayed onto a skin surface in a manner like sandblaster, CO2 laser and a method for transpiration of surface tissues of a living body using Erbium YAG laser have been known. Further, in the field of cosmetics, methods of removal of waste product or aging keratin by hand-massage (for example, refer to a patent literature, Japanese Publication No.56-131512) after providing inorganic abrasive particles of a few micrometers to tens of micrometers onto a skin surface in order to smooth and cleanse the skin surface are also known.

However, among the peeling techniques described above, tape peeling is not only the most primitive method but also a method carried out at a low cost and with ease. Nevertheless, the method has disadvantages that it cannot peel a barrier on the skin surface uniformly and accurately as well as it accompanies pain.

A method of transpiration of the skin surface tissues using laser is capable of peeling a barrier on the skin surface uniformly and accurately, but it requires an expensive device.

A method of abrasion cannot peel a barrier on the skin surface uniformly and accurately as well as it requires an expensive device.

Compared with those methods, a method using inorganic abrasive particles with an average particle diameter of a few micrometers to tens of micrometers is preferable in terms of accompanying no pain and being carried out at a low cost and with ease. However, it cannot peel the barrier on the skin surface uniformly and accurately. That is, if the abrasive particles have an average particle diameter larger than a few micrometers, the particle diameter is too large to remove the barrier on the skin surface uniformly and accurately because an average thickness of a barrier on the skin surface is approximately a few micrometers to tens of micrometers. In this case, if the barrier on the skin surface is removed, the skin surface may be peeled off more than required, giving rise to damage to healthy tissues and an enhanced risk of germ infection. Moreover, when hand-massage is carried out, it is difficult to provide uniform pressure onto the skin surface, resulting in additional difficulty in uniform and accurate peeling.

In addition to that, if the particles of the order of micrometers are not cleansed off after being used, a foreign-body sensation may persist when the particles remain on the skin surface or get onto the surface of the eyeball or into the mouth. In particular, there may be a risk that sharp edges of the particles stick to the skin tissues or mucous membrane and cannot be taken out in some cases depending on the forms of the particles. In such cases, there may be not only a persistent sensation of the foreign substance after use but also a risk for facilitating damage and infection of the tissues.

Accordingly, the primary object of the present invention is to provide a technique by which a barrier on the skin surface can be peeled off uniformly and accurately at a low cost, with ease and without accompanying any pain.

### SUMMARY OF THE INVENTION

The present invention that has solved the above problems is as follows:

A first feature of the present invention is a delivery method of a substance via skin absorption, characterized in that abrasive particles having an average particle diameter smaller than 1000 nm are used for peeling off a skin surface, while a delivery substance is provided to the skin surface and the delivery substance is to be delivered to the inside of skin via skin absorption from the peeled skin surface.

As described above, a thickness of a barrier on the skin surface is as small as a few micrometers to tens of micrometers. Therefore, it is possible to peel off the barrier in stages by using abrasive particles having an average particle diameter smaller than 1000 nm according to the present invention. Thus, it is possible to peel the skin surface uniformly and accurately as well as to control levels enough for removal of the barrier on the skin surface, thereby decreasing in the risk of bacterial infection and the like.

In the first feature, peeling is performed by giving vibration with an amplitude of 10 μm to 1000 μm and a frequency of 1 kHz to 10 MHz to the abrasive particles, the delivery substance and the skin surface in a state where the abrasive particles and the delivery substance are both provided to the skin surface. It is preferable to perform peeling and delivery at the same time. It is a matter of course that uniform, accurate and high-speed peeling is possible by providing fine and high-speed vibration as described above. However, that is not all of the first feature. In other words, the skin absorption takes place through pores on the skin surface. The absorption efficiency mainly depends on activation of the tissue of the skin surface and probability of reaching of the substance to absorption pores. In addition, when high-speed and fine vibration is used as described above, not only are tissues of the skin surface activated but also probability of reaching of the delivery substance to the absorption pores is enhanced by fine vibrating motion of the delivery substance, and expansion, shrinkage and fine move of the absorption pores by the vibration. As a result, absorption of the delivery substance is accelerated as well as uniform, accurate and high-speed peeling is facilitated. It should be noted that lowering the amplitude to smaller than 10 μm is not preferable, because the abrasive particles and the delivery substance are hardly vibrated by an amplitude smaller than 10 μm, which leads to decrease in the efficiency of peeling and delivery. In addition to that, when the amplitude exceeds 1000 μm, the peeling becomes too rough, giving rise to difficulty in accurate peeling.

It is possible to select abrasive particles appropriately for the present invention depending on kinds of the delivery substance and objects of the delivery. It is preferable, in particular, to use globular silica particles. The advantages of using silica particles are as follows; silica particles are biologically nontoxic, silica particles keep transparency without changing their color even when they are applied to skin or mixed with cosmetics or body soap, and globular silica particles of the order of nanometers which hardly give damage to skin are relatively obtainable with ease. Particularly, not only does uniform and accurate peeling become possible with the particles in a uniform form but also the globular particles are suitable for being left on the skin during the delivery of the substance because of less discomfort caused by the residual substance foreign to the skin and the like. Further, the globular particles do not have any sharp edges, which leads to less fear that it becomes impossible for the particles sticking to the skin tissue or mucous membrane to be taken out. Furthermore, there is less risk of causing the tissues to be damaged and of facilitating the tissues to be infected.

In addition, as described above, it is preferable to use particles having an antibacterial property as the abrasive particles of the present invention because when the skin surface is peeled off, absorption of the delivery substance is accelerated and absorption of undesired bacteria is accelerated as well.

It is preferable to use particles having an average particle diameter smaller than 100 nm as the abrasive particle for the present invention. By using abrasive particles having such an average particle diameter, it is possible to peel more uniformly and more accurately. Among such abrasive particles, abrasive particles having a particle diameter smaller than 10 nm, in particular, are trapped in the patterned indented surface of cuticle after scrubbing and retained in the cuticle until metabolic exfoliation of the cuticle occurs (it is said that it usually takes a few days for metabolic exfoliation to occur). Therefore, when particles, for example, antibacterial particles or the like having functions besides that of scrubbing are used, the functions persist for a long time.

Moreover, a medical substance and/or a cosmetic substance may be used as the delivery substance of the present invention. According to the present invention, such substances are made possible to be delivered into the inside of skin at a low cost and with ease without giving any pain.

On the other hand, the first feature can also provide the skin surface with an antibacterial agent, a disinfectant and/or a germicide besides the abrasive particles and the delivery substance.

Next, a second feature of the present invention is a peeling agent characterized in that the peeling agent used for delivering a substance via skin absorption contains, in a dispersed medium, abrasive particles having an average particle diameter smaller than 1000 nm and a delivery substance to be delivered into the inside of the skin via skin absorption. In this case, abrasive particles are preferably globular silica particles. It is also preferable that the abrasive particles have an antibacterial property. In addition, it is preferable for the abrasive particles to have an average particle diameter smaller than 100 nm. It is preferable for the delivery substance to include a medical substance and/or a cosmetic substance as well. Further, it is preferable to contain an antibacterial agent, disinfectant and/or germicide besides the abrasive particles and the delivery substance. It should be noted that the effects and advantages of those constructions have been described in the first feature, so that explanation for them will be omitted in order to avoid repetition.

Next, a third feature of the present invention is characterized by a peeling device comprising a grip portion and a pad portion attached to the grip portion, wherein the pad portion has an abrasive pad, a storage where a peeling agent containing at least abrasive particles is stored, and a supply path for supplying the peeling agent stored in the storage to the abrasive pad. A user holds the grip portion, and presses and rubs the abrasive pad against the skin, thereby carrying out peeling. Further, it is easy to supply the peeling agent because the peeling agent stored in the storage is supplied to the abrasive pad through the supply path.

In the peeling device of the present invention, the peeling device is preferably provided with the grip portion having a pad attachment portion connected through an elastic connecting member, the pad attachment portion having a means for generating vibration, wherein the pad portion is attached to the pad attachment portion. If the pad attachment portion of the grip portion and the pad portion are connected to each other through the elastic connection member as described above, the pad attachment portion and the pad portion are freely movable within the range of deformation of the elastic connecting member even though the grip portion is held tight. Accordingly, even in a state where a user hold the grip portion, the vibration generated by the means for generating vibration which is built in the pad attachment portion is not restrained, the pad attachment portion and the pad portion are vibrated against the grip portion, and thus efficient peeling is possible.

In the peeling device of the present invention, the abrasive pad has a flat front-end face and the means for generating vibration preferably generates vibration in a direction substantially parallel to the front-end face of the abrasive pad. In particular, the vibration may be linear reciprocatory vibration; however, the vibration is preferably rotary vibration. By employing such configuration of rotary vibration, it is possible to peel uniformly and accurately.

In addition, for the peeling device of the present invention, the peeling agent preferably contains abrasive particles having an average particle diameter smaller than 1000 nm, an amplitude of the means for generating vibration is preferably 100 μm to 1000 μm, and a frequency is preferably 1 kHz to 10 MHz.

As a means for generating vibration, there may be various configurations conceivable. When improvement in peeling efficiency by high frequency is taken into consideration, it is preferable to use an ultrasound oscillator. However, the amplitude of the ultrasound oscillator is small, and the peeling efficiency is not enhanced in spite of the frequency. For this reason, it is preferable to provide an amplification means for amplifying the vibration in the process of transmitting the vibration generated by the ultrasound oscillator to the abrasive pad.

Further, for the peeling device of the present invention, it is preferable for the peeling agent to contain a delivery substance to be delivered inside the skin via skin absorption.

Furthermore, the following is proposed as a more preferable configuration for the peeling device of the present invention:

wherein the pad portion has a front end and a base end, a tubular member provided with a partition wall in the middle thereof in the longitudinal direction, and a piston arranged on the base-end side of the partition wall within the tubular member,

wherein the abrasive pad has a front-end portion and a base-end portion; the base-end portion is retained on the front-end portion of the partition wall within the tubular member; and the front-end portion protrudes from the front end of the tubular member,

wherein a part between the partition wall and the piston serves a storage,

wherein a supply path passes through the partition wall and the abrasive pad and has an opening on the outer face of the front-end portion of the abrasive pad, and

wherein a grip portion has a front-end portion and a base-end portion, the front-end portion of the grip portion is inserted into the base-end side of the partition wall within the tubular member, and the peeling agent within the storage is supplied to the outer face of the front-end portion of the abrasive pad through the supply path by inserting the piston with the front-end portion of the grip portion.

With this configuration, when a user holds the grip portion and presses the pad portion against the skin, the piston is inserted by the front-end portion of the grip portion, and then the peeling agent within the storage is supplied to the outer face of the front-end portion of the abrasive pad through the supply path. Consequently, the peeling agent can be supplied to the abrasive pad with ease according to user's need.

Further, for the peeling device of the present invention, it is preferable to attach a pad portion detachably to the grip portion so that the pad portion may be disposed.

On the other hand, a fourth feature is associated with only the pad portion described above and is a disposable pad for the peeling device, wherein the pad portion has a front end, a base end and a tubular member provided with a partition wall in the middle thereof in the longitudinal direction,

wherein a piston is arranged on the base-end side of the partition wall within the tubular member,

wherein an abrasive pad has a front-end portion and a base-end portion; the base-end portion is retained on the front-end side of the partition wall within the tubular member; and the front-end portion protrudes from the front end of the tubular member,

wherein a storage for a peeling agent is formed as a space between the partition wall and the piston,

wherein a peeling agent fills this storage and contains at least abrasive particles,

wherein a supply path for the peeling agent extending from the storage for a peeling agent through the partition wall and the abrasive pad to an opening on the outer face of the front-end portion of the abrasive pad, and

wherein a grip portion is configured to be inserted removably on the base-end side of the partition wall within the tubular member.

By using such a disposable pad, supply of a peeling agent is facilitated as well as clean peeling is possible since a delivery substance, for example, can be sterilized under tight sealing and the pad can be disposed according to each individual subject or site for peeling.

### BRIEF DESCRIPTION OF THE DRAWINGS

FIG. 1 is a longitudinal sectional view showing a first embodiment of a peeling device.

FIG. 2 is a perspective view showing the first embodiment of the peeling device.

FIG. 3 is a broken exploded view of a main part of a pad portion.

FIG. 4 is an explanatory diagram of the peeling device in use.

FIG. 5 is a longitudinal sectional view showing a second embodiment of the peeling device.

FIG. 6 is a top plan view showing a principle of generation of vibration.

FIG. 7 is an enlarged view of the main part.

FIG. 8 is an explanatory diagram of the peeling device in use.

FIG. 9 is a longitudinal sectional view showing the second embodiment of the peeling device.

FIG. 10 is a broken perspective view of a main part showing the second embodiment of the peeling device.

FIG. 11 is a top plan view showing a principle of generation of vibration.

FIG. 12 is a schematic view for explaining differences of abrasive effects among forms of particles; and

FIG. 13 is a schematic view for explaining a delivery effect by porous particles.

### PREFERRED EMBODIMENTS OF THE INVENTION

Hereinafter preferred embodiments of the invention will be described in detail.

(Delivery Method and Peeling Agent)

In a delivery method of the present invention, abrasive particles having an average particle diameter smaller than 1000 nm are used for peeling a skin surface, while a delivery substance is provided onto the skin surface and the delivery substance is delivered from the peeled skin surface into the inside of the skin via skin absorption. Accordingly, after providing the abrasive particles onto the skin surface to peel the skin surface, the abrasive particles are either removed or left there and then a delivery substance can be provided onto the skin surface. On the other hand, it is also possible to provide both abrasive particles and a delivery substance onto the skin surface, followed by carrying out peeling and skin absorption of the delivery substance at the same time.

At the time of carrying out peeling, it may be possible for the abrasive particles to be scrubbed on the skin by hand-massage. However, it is preferable to use a peeling device described below in order to carry out peeling uniformly and accurately.

Delivery substances for the present invention are not limited in particular as long as the substances are used for the purpose of is delivered into the inside of skin via skin absorption. Medical substances and/or cosmetic substances, for example, anesthetics, vaccines, analgesics, anti-inflammatory agents, germicides, antibacterial agents, bacteria removing agents, disinfectants, hair restoration tonics, collagen, various kinds of nutrients (vitamins C, E, A and the like) and the like can be used. Of course, only one of the above substances can be used in the present invention or multiple kinds of them can be selected appropriately for use. According to the present invention, a medical substance or a nutritional substance conventionally delivered by, for example, injection into the inside of skin can be delivered into the inside of skin not only without giving any pain but also with ease and at a low cost, which significantly facilitates administration of, for example, vaccine and the like to children.

Abrasive particles used for the delivery method of the present invention can be appropriately selected depending on the kind of the delivery substance and the object of the delivery; however, it is preferable to use silica particles, in particular. Silica particles are hard and have no effects on almost all the delivery substances. Moreover, silica particles are biologically nontoxic. When peeling is carried out in a state where both a delivery substance and abrasive particles are provided to skin, the abrasive particles must be left on the skin until the delivery is completed or becomes saturated. Therefore, if the delivery substance is a colored substance such as titanium dioxide, it is inconvenient to resume ordinary daily work immediately after peeling. Further, when abrasive particles are provided in a form of cosmetics or body soap, it becomes difficult to adjust colors of the cosmetics and the like as desired if the abrasive particles are made of a colored substance such as titanium dioxide. On the other hand, silica particles are preferable because clear and colorless silica particles are obtainable. Furthermore, there is an advantage that globular silica particles of the order of nanometers which hardly give damage to skin are obtainable relatively with ease.

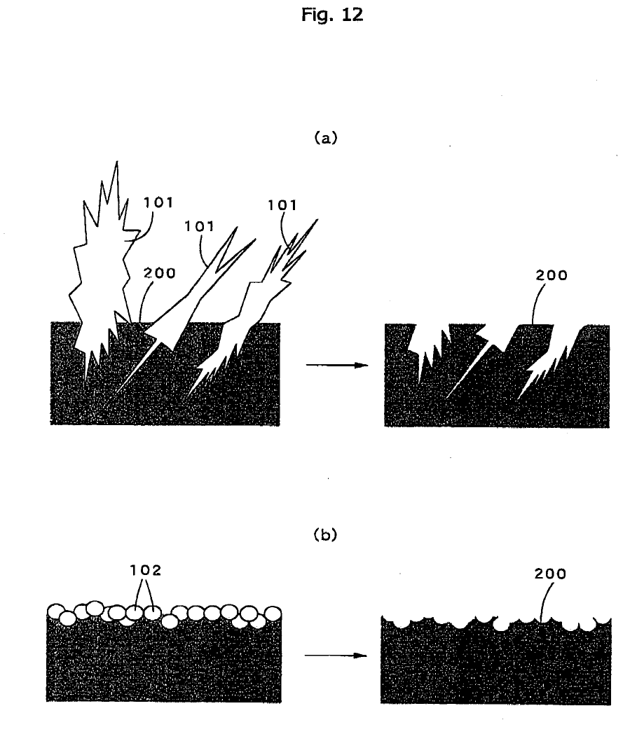

As a form of abrasive particles, a globular form that hardly gives damage to skin is preferable. However, particles in other forms such as a stick form and the like may be permissible. That is, as shown in FIG. 12(a), it is basically difficult for particles having irregular-shaped cross sections in a needle-like shape 101 and the like to scrub a skin surface 200 uniformly and accurately. In addition, there is a risk to give damage to the tissues in the layer lower than cuticle. Besides the above, there are other risks that sharp edges of the particles stick to the skin tissues and mucous membrane and cannot be pulled out of them, leading to discomfort caused by the substance foreign to the skin which remain after the use as well as facilitation of the tissues to be damaged and infected. Contrary to the particles having sharp edges, globular particles 102 as shown in FIG. 12(b) have less risk to scrub the skin surface 200 too deep. Therefore, it is easy for the particles to remove a barrier on the skin surface uniformly and accurately, and there is also less risk for the particles to stick into the tissues, skin tissues and mucous membrane and to become unremovable.

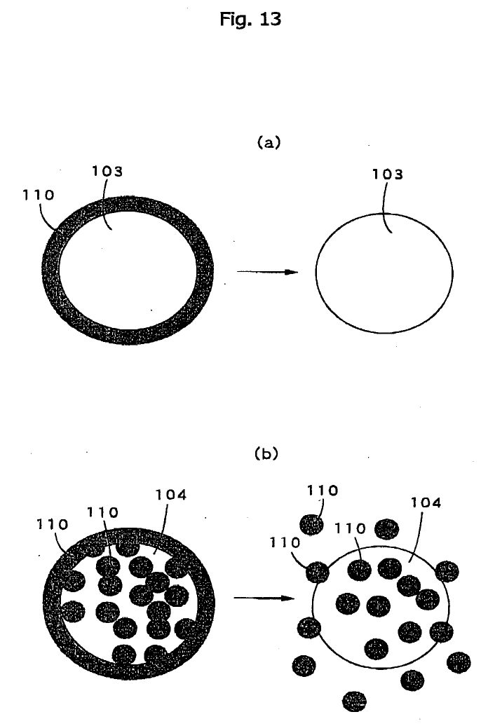

In terms of a retained amount of the delivery substance, abrasive particles are preferably porous particles having a large surface area 104 as shown in FIG. 13(b). With the use of these particles, there are advantages that not only does the retained amount of a delivery substance 110 increase but also the amount and the velocity of discharge of the delivery substance 110 can be controlled by varying pore sizes, forms, and molecular weight, forms, hydrophilicity, lipophilicity, kinds of solvents and the like of medical substances and cosmetics. Thus, there are advantages that the delivery effect lasting long can be achieved and so on. Contrary to the above particles, with the use of non-porous particles 103 as shown in FIG. 13(a), the delivery substance 110 retained only on the surface can be discharged at once. Therefore, the non-porous particles are suitable in cases where only a rapid effect is expected and so on. Moreover, if the non-porous particles are globular, they have an adequately large surface area to retain a sufficient amount of the delivery substance 110.

On the other hand, although absorption of the delivery substance is facilitated when a skin surface is peeled, a risk of bacterial infection increases. Accordingly, it is preferable to use only abrasive particles having an antibacterial property such as those made of silver complex, silica containing silver, apatite and the like, or to use the abrasive particles mixed with silica particles. However, it should be noted that globular forms of those particles are not readily obtainable and that there is a risk for the delivery substance to be oxidized by silver depending on the kinds of delivery substances.

For the size of the abrasive particles, an average particle diameter is at least smaller than 1000 nm. It is possible to peel off a barrier in stages by using such abrasive particles because a thickness of the barrier on the skin surface is as small as a few micrometers to tens of micrometers. Thus, it is possible to peel off uniformly and accurately, thereby controlling levels adequately enough for removal of the barrier on the skin surface and decreasing in the risk of bacterial infection and the like. From this viewpoint, amore preferable size of abrasive particles has an average particle diameter smaller than 100 nm.

Among such abrasive particles, abrasive particles having a particle diameter smaller than 10 nm, in particular, are trapped in the patterned indented surface of cuticle after scrubbing and retained in the cuticle until metabolic exfoliation of the cuticle occurs (it is said that it usually takes a few days). Therefore, when particles, for example, antibacterial particles or the like having functions other than scrubbing are used, those functions are exerted for a long time. If particles are silica particles, it is possible to manufacture particles having an average particle diameter in the range of a few nanometers to tens of nanometers. From the above viewpoints, preferable abrasive particles, in particular, are globular silica particles having an average particle diameter of 1 nm to 100 nm.

Although abrasive particles and a delivery substance may be applied onto skin directly, it is preferable to make them in a form of gel, cream, solution, emulsion, lotion, body soap (liquid/solid soap, shampoo and the like) or the like containing both of them or just one, thereby applying it onto the skin.

In the delivery method of the present invention, a method for providing a scrubbing force for the abrasive particles is not particularly limited. However, when a size of the abrasive particle is taken into consideration, it is preferable to provide vibration with an amplitude of 100 μm to 1000 μm and a frequency of 1 kHz to 10 MHz. It is possible to peel uniformly, accurately and speedily by providing fine and high-speed vibration as described above.

In a more preferable feature, peeling is carried out by providing the abrasive particles, delivery substance and skin surface with vibration with an amplitude of 100 μm to 1000 μm and a frequency of 1 kHz to 10 MHz in a state where the abrasive particles and delivery substance are applied onto the skin surface, and the peeling and delivery are carried out at the same time. In this case, not only are the tissues around the skin surface activated by high-speed and fine vibration but also probability of reaching of the delivery substance to absorption pores is enhanced by fine movement of the delivery substance, as well as expansion and fine movement of the absorption pores induced by the vibration. As the result, it is possible not only to peel uniformly, accurately and speedily but also to enhance absorption of the delivery substance.

Further, in the delivery method of the present invention, regardless of whether or not antibacterial abrasive particles are used, an antibacterial agent, disinfectant and/or germicide may be provided to the skin surface separately from the abrasive particles and the delivery substance.

(Peeling Device)

Next, a peeling device of the present invention will be described hereinafter. The peeling device of the present invention is preferable for delivery of such a substance as described above. In addition, the device is usable in peeling for just removal of cuticle or the like without carrying out delivery of a substance.

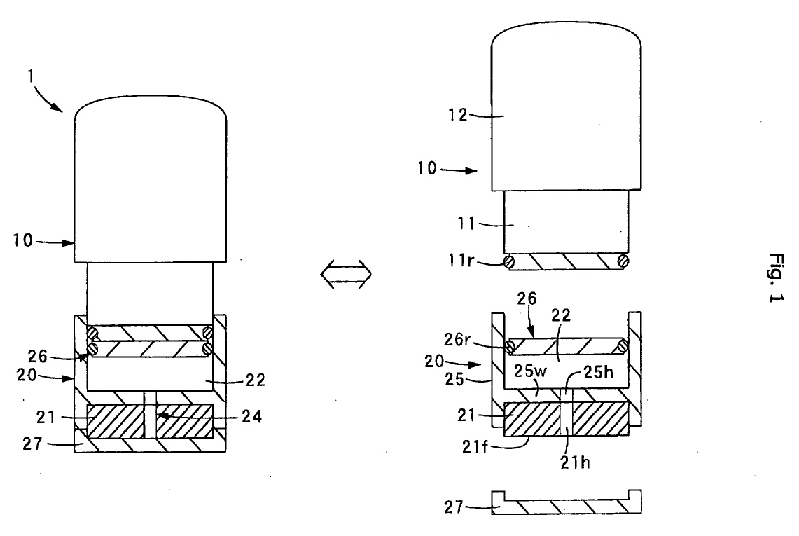

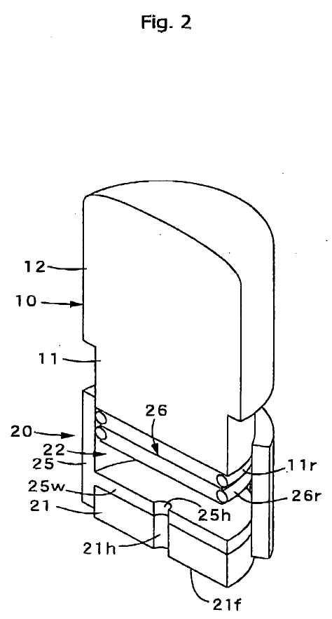

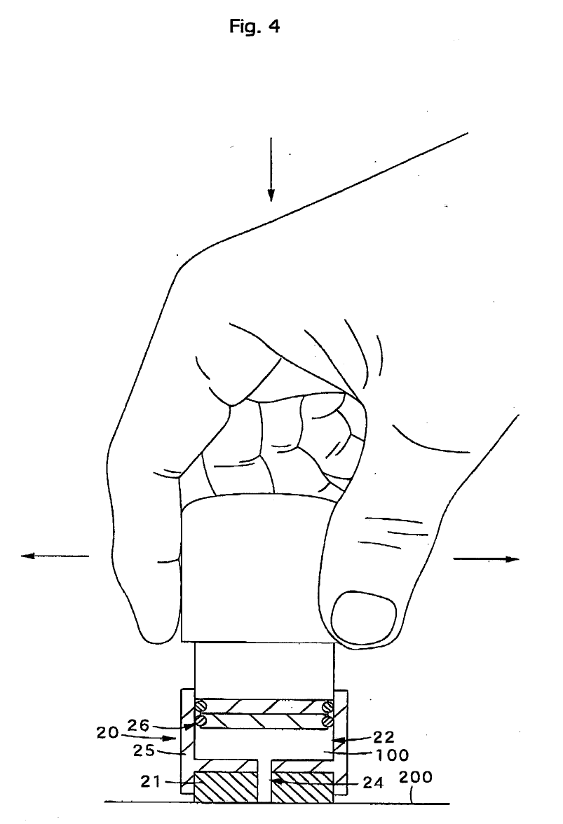

FIG. 1, FIG. 2 and FIG. 4 show a first embodiment of the peeling device associated with the present invention. The peeling device comprises a grip portion 10 in a cylindrical shape having a front-end portion 11, a base-end portion 12, and a pad portion 20 attached to the grip portion 10.

The pad portion 20 is composed of an abrasive pad 21, a storage 22 for storing a peeling agent containing at least abrasive particles and a supply path 24 for supplying the abrasive pad 21 with a peeling agent 100 stored in the storage 22. The arrangement and the like of the construct can be varied in various ways as long as all the components are included.

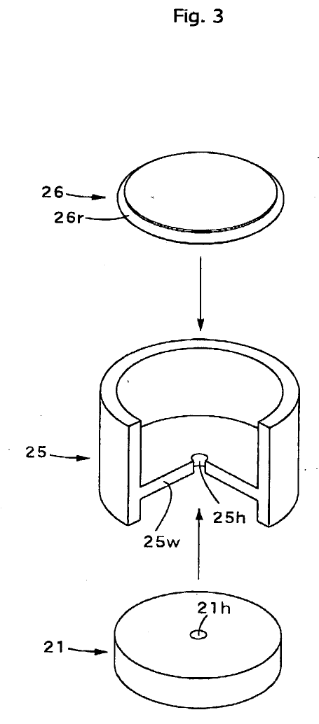

The pad portion 20 of the present first embodiment is composed of a tubular member 25, a piston 26 and the abrasive pad 21 as shown in FIG. 3. The tubular member 25 is a cylindrical member having a front end and a base end as well as a partition wall 25w provided in the middle thereof in the longitudinal direction. A through-hole 25h communicating the front-end side with the base-end side is formed at the center of the partition wall 25w. One through-hole 25h may be formed as illustrated; however, a plurality of through-holes may also be formed. Further, a location for forming the through-hole 25h is not limited to the center and can be arranged as desired. If a plurality of the through-holes 25h are provided, they may be arranged in line.

The piston 26 is a discal member to be inserted into a space on the base-end side of the partition wall 25w inside of the tubular member 25. A ring-shaped seal member (so called O-ring) 26r is attached on the face of the outer circumference of the piston 26. This seal member 26r is configured to air tightly contact with the face of the inner circumference of the tubular member 25.

The grip front-end portion 11 is removably inserted into the base-end side of the piston 26 inside of the tubular member 25 in the pad portion 20. Because of this configuration, the grip front-end portion 11 is in a cylindrical shape and a ring-shaped elastic member (so called O-ring) 11r is attached on the face of the outer circumference thereof. This elastic member 11r is in close contact with the face of the inner circumference of the tubular member 25 in the pad portion 20, thereby fixing the grip front-end portion 11 removably to the pad portion 20.

On the other hand, the abrasive pad 21 has a front-end portion and a base-end portion. The base-end portion is inserted into and retained on the front-end side of the partition wall 25w inside of the tubular member 25 as well as the front-end portion is protrudes from the front end of the tubular member 25. The abrasive pad 21 in the example illustrated is in a discal shape, having a flat front-end face 21f and a flat base-end face, and a through-hole 21h is formed at the center thereof. For the abrasive pad 21, an abrasive pad made of a deformable porous member, e.g. aggregation of fiber such as felt, a resin and the like is preferably used so that an abrasive agent and the like may be retained therein and a close contact property with skin may be furnished. The abrasive pad 21 which has less continuous bubbles and does not allow enough permeation of peeling agent may be acceptable. However, a preferable abrasive pad 21 has a high proportion of continuous bubbles and allows permeation of the peeling agent in some measure. In the latter case, such a through-hole 21h as in the illustrated example may be omitted. The abrasive pad 21 is preferably fixed undetachably to the tubular member 25 of the pad portion by adhesion or the like. However, in order to make the abrasive pad 21 alone replaceable, it is preferable to employ a detachably fixed configuration such as interdigitation, screwing together and the like.

The space between the partition wall 25w and the piston 26 in the pad portion 20 serves as the storage 22 for a peeling agent. The storage 22 is filled with a peeling agent containing at least abrasive particles. As the peeling agent, it contains at least abrasive particles, and a form of liquid or fluid in which the peeling agent is supplied from the storage 22 to the abrasive pad 21, for example, gel, cream, solution, emulsion, lotion, liquid soap and the like can be employed. Further, when the present device 1 is used for the purpose of delivery of a medical substance or a cosmetic substance into the inside of the skin as described before, it is permissible for those delivery substances to be applied to the skin separately but it is preferable to use them in a mixture with the peeling agent.

For use of the peeling device configured as described above, a user holds the grip portion 10 as shown in FIG. 4, presses and rubs the abrasive pad 21 against a skin surface 200, thereby scrubbing the skin surface 200 with the abrasive particles existing between the skin surface 200 and the abrasive pad 21. Accordingly, waste product, aging keratin and the like are removed. If the front-end face 21f of the abrasive pad is flat as in the configuration illustrated, uniform pressure can be applied to a wider area compared with that applied by hand-massage, giving rise to more accurate peeling. Further, the user can press the grip portion 10 hard against the skin according to his or her need, thereby inserting the piston 26 of the pad portion 20 into the inside of the tubular member 25 by the frond end portion 11 of the grip portion 10. Consequently, the peeling agent 100 within the storage 22 is pushed out through the supply path 24 composed of a through-path 25h of the partition wall 25w and a through-path 21h of the abrasive pad 21, thereby supplying the peeling agent to the front-end face 21f of the abrasive pad. At this time, when the abrasive pad 21 allows permeation of the peeling agent, the peeling agent is permeated and retained inside of the abrasive pad, and then it is supplied to the front-end face 21f of the abrasive pad by deformation of the abrasive pad 21. Accordingly, it is possible to supply the peeling agent with ease to the abrasive pad 21 in response to the user's need.

Furthermore, the pad portion 20 is detachably attached to the grip portion 10. Therefore, it is easy to detach the pad portion 20 and the pad portion 20 can also be made disposable. In this case, a disposable pad portion is highly suitable for carrying out administration of, for example, vaccine and the like in the delivery method described above.

In a more preferable configuration, a cap 27 can be attached to cover the whole exposed portion of the abrasive pad 21 in the pad portion. A user can remove the cap 27 before use and attaches the cap 27 after use, which makes it possible to use the abrasive pad 21 cleanly. In particular, when the pad portion 20 is disposable as described, sealed sterilization process can be carried out by covering the front end of the tubular member 25 by the cap (the base-end side is sealed by the piston 26).

In the first embodiment 1 described above, a user moves the peeling device 1 manually. Therefore, even though more accurate scrubbing is possible compared with that by hand-massage, the accuracy is limited and its efficiency is not adequate. Thus, the present invention proposes a second and third embodiments.

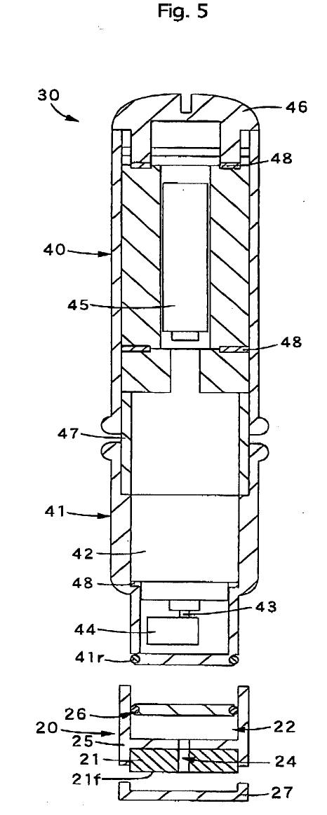

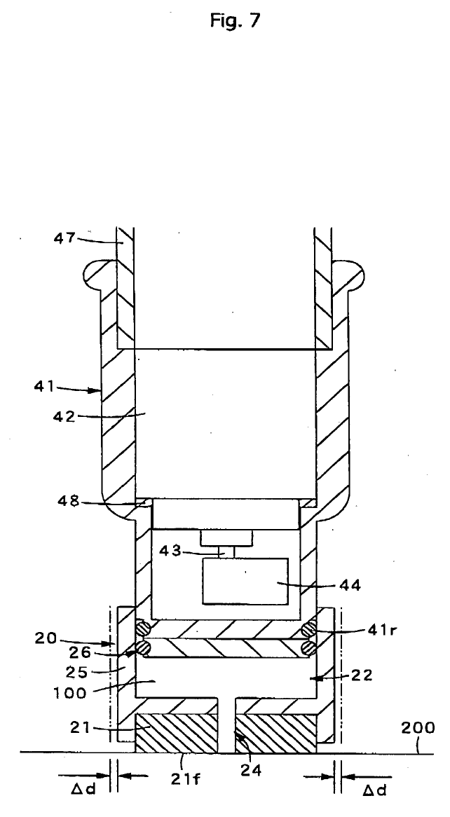

FIGS. 5, 7 and 8 show a motor-driven vibration type of peeling device 30 associated with the second embodiment. The peeling device 30 is composed of a tubular grip portion 40 having a pad attachment portion 41 on the front end thereof and a pad portion 20 attached to the pad attachment portion 41. The pad portion 20 is the same as that in the first embodiment. The pad attachment portion 41 of the grip portion 40 is detachably inserted into the inside of the base-end side of the piston 26 of the tubular member 25 in the pad portion 20. For this reason, the pad attachment portion 41 has a front end and a base end in a cylindrical shape, and a ring-shaped elastic member 41r is attached on the face of the outer circumference thereof. The ring-shaped elastic member 41r is in close contact with the face of the inner circumference of the tubular member 25 of the pad portion 20, giving rise to detachable attachment of the grip portion 40 to the pad portion 20.

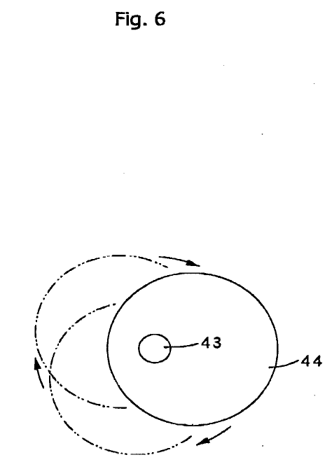

In the inner space of the pad attachment portion 41 of the grip portion 40, a means for generating vibration which is composed of an electric motor 42 and an eccentric plumb 44 attached to a rotary shaft 43 of the electric motor 42 is fixed. The center of gravity of the eccentric plumb 44 is eccentric of the rotational center of the rotation shaft 43. As shown in FIG. 6, when the eccentric plumb 44 is rotated by the motor 42, the center of gravity of the eccentric plumb 44 moves toward the rotation direction, giving rise to generation of rotary vibration which is parallel to a plane orthogonal to the center of gyration of the motor 42 and is coaxial to the center of gyration of the motor 42. The vibration generated is transmitted to the pad portion 20 through the pad attachment portion 41. In the example illustrated, the direction of the center of gyration of the motor 42 and the direction of the central axis of the pad attachment portion 20 are coaxial. The center of gyration of the motor 42 is designed to be orthogonal to a front-end plane 21f of the abrasive pad 21. For this reason, the rotary vibration which is substantially parallel to the front-end face 21f of the abrasive pad and is coaxial to the axis orthogonal to the front-end face 21f of the abrasive pad is to be generated.

An amplitude Δd of this means for generating vibration can be determined appropriately depending on weight, size and eccentric degree of the eccentric plumb 44. In addition, a frequency can also be determined appropriately depending on the number of revolutions of the motor 42. It is preferable for the amplitude Δd to be determined according to the size of the abrasive particles contained in the peeling agent. When the average particle diameter of the abrasive particles is smaller than 1000 nm, 100 μm to 1000 μm is preferable. The frequency is preferably as high as possible because frequency affects the peeling efficiency (when a substance is delivered, the frequency affects the delivery efficiency as well.). However, when the electric motor 42 driven by a battery 45 and obtainable at a low cost is used, the frequency is preferably as high as 1 kHz to 100 kHz.

It may be acceptable for power of the electric motor 42 to be supplied through a power cable from external power source equipment and to be turned on/off by a foot pedal switch or the like. In order to make the configuration simpler as shown in the examples illustrated, a power supply such as a dry cell 45, a battery and the like is made built-in in the space of the grip portion 40, the base end portion of the grip portion 40 is constructed as a cap portion 46 for opening and closing the grip portion at the time of replacing a battery and the like, the power supply 45 and motor 42 are connected by a power cable not shown (because of this, the grip portion 40 and an elastic connecting member 47 described later are made tubular), and a switch 46 is provided on the outer face of the grip portion 40 for starting and stopping rotation of the motor 42.

In a preferred configuration, the pad attachment portion 41 is connected to the grip portion 40 via the elastic connecting member 47 as illustrated. Any material is good for the elastic connecting member 47 as long as it is hard to transmit vibration and is capable of vibrating the pad attachment portion 41 freely against the grip portion 40. Therefore, a flexible tube in an accordion shape made of rubber, for example, can preferably be used. Besides this, it is preferable to sandwich vibration-absorbing materials 48 between the component members for controlling the undesired transmission of vibration as illustrated, and to prevent chatter generation, wear-out and a device failure.

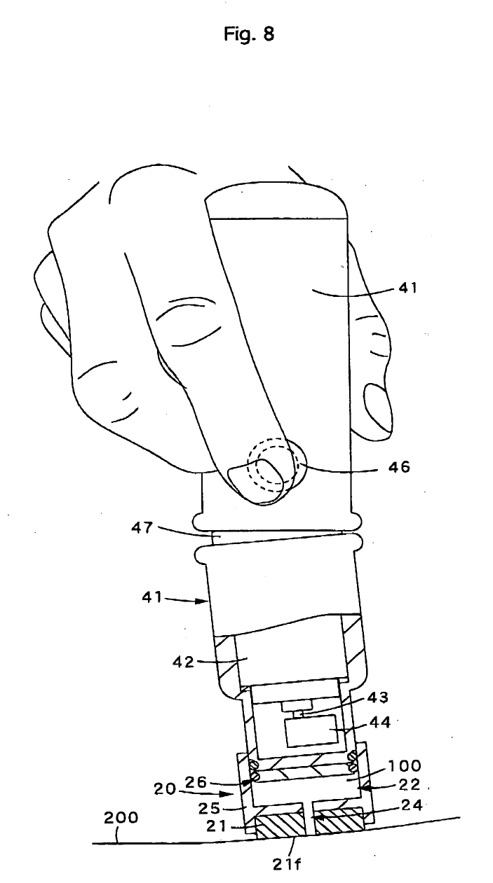

When the peeling device 30 of the second embodiment as constructed above is used, a user holds the grip portion 40 as shown in FIG. 8, and presses the abrasive pad 21 against the target area of the skin surface 200 as the user drives and stops the means for generating vibration with manipulation of the switch 46 as desired. The vibration generated by the means for generating vibration is transmitted to the abrasive pad 21 and then the skin surface 200 is scrubbed by the abrasive particles existing between the skin surface 200 and the abrasive pad 21, thereby removing waste product, aging keratin, and the like. Particularly, the abrasive pad 21 is rotationally vibrated parallel to the front-end plane 21f and coaxially to the axis orthogonal to the front-end plane 21f. For this reason, it is capable of remarkably uniform and accurate scrubbing and preventing irregular peeling, which is different from a case where only linearly reciprocating vibration is applied. In addition, when such rotary vibration is applied to the delivery method of substances of the present invention described above, the vibration area of the delivery substance, the expanding/shrinking area of the absorption pores and the moving area increases compared with those by linear vibration, giving rise to enhancement in probability of reaching of the delivery substance to the absorption pores.

Further, if the pad attachment portion 41 is being connected to the grip portion 40 via the elastic connecting member 47 as in the configuration illustrated, not only is it hard for vibration generated by the means for generating vibration to be transmitted to the user's hand but also the pad portion 20 comes to vibrate freely against the grip portion 40, thereby preventing transmission loss of the vibration. Furthermore, in this case, not only is it possible for the pad attachment portion 41 and the pad portion 20 to be bent in all the directions to the grip portion 40 but also it is possible for the abrasive pad 21 to be deformed. Therefore, the user can move the abrasive pad 21 along the curve of the chin, the scalp and the like as maintaining close contact with the abrasive pad 21 and the skin surface 200.

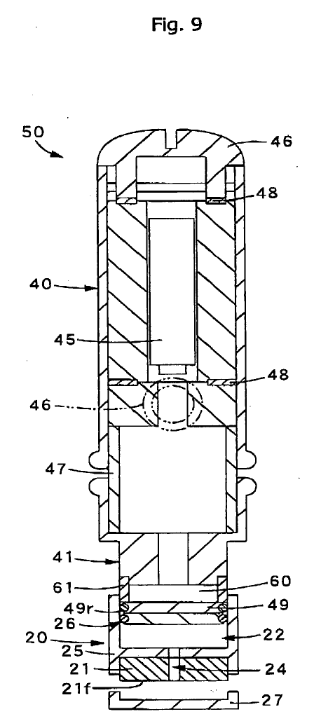

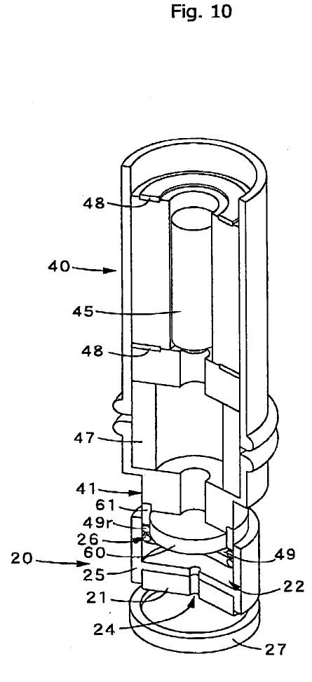

A third embodiment is provided with improved peeling efficiency more than that of the second embodiment (when a substance is delivered, the delivery efficiency is also improved). That is, as illustrated in FIG. 9 and FIG. 10, a peeling device 50 in the third embodiment employs an ultrasound oscillator 60 as a means for generating vibration, and other composition and effects are substantially the same as those in the second embodiment described before.

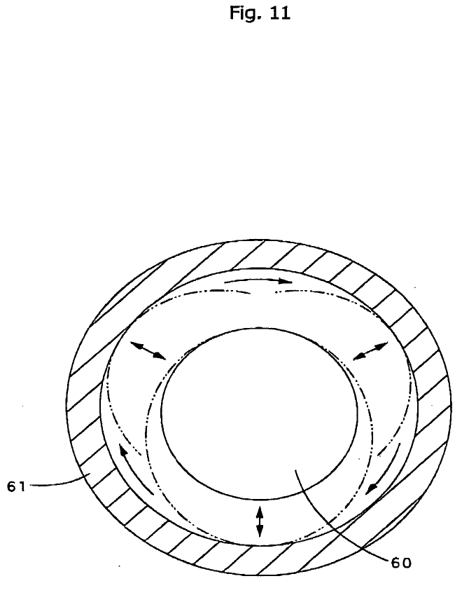

A common ultrasound oscillator which reciprocatingly vibrates only in one axial direction can be used as the ultrasound oscillator 60. However, a vibration type which vibrates orthogonally in biaxial directions respectively (referred to as orthogonal biaxial vibration type hereinafter) can be used preferably. When an orthogonal biaxial vibration type is used, it is possible to create various vibration patterns, as a whole oscillator, by controlling vibration in each axial direction. An especially preferable pattern is shown in FIG. 11. The ultrasound oscillator 60 generates vibration by projecting deformation of piezoelectric element (arrows outward from the center show this deformation in the illustration). This deformation is generated in turn in rotation directions, centering the point of intersection of the orthogonal biaxes (arrows in a periphery direction show this deformation in the illustration), thereby allowing rotary vibration to be generated substantially parallel to the plane formed by the two axes orthogonal to each other.

In the illustrated example, the ultrasound oscillator 60 of the orthogonal biaxial vibration type is built-in in the pad attachment portion 41 so as to make the plane formed by the two axes orthogonal to each other, parallel to the front-end plane of the abrasive pad 21. Therefore, in this case, on transmitting the rotary vibration generated by the ultrasound oscillator 60 to the abrasive pad 21, the abrasive pad 21 is rotationally vibrated around the axis orthogonal to the front-end plane thereof.

On the other hand, the frequency of the ultrasound oscillator 60 can be raised up to the order of MHz, and the vibration generated is effectively transmitted as it is; however, a frequency of an ultrasound oscillator of the order of MHz results in the amplitude as small as tens of micrometers. For this reason, peeling efficiency cannot be improved in spite of the improvement in the frequency.

Here, a means for amplifying the vibration generated by the ultrasound oscillator 60 in the process of transmitting the vibration to the abrasive pad 21 is proposed as an especially preferable configuration. As such an amplification means, for example, a movable amplifying member 61 can be arranged independently of the oscillator 60 and the pad portion 21 to be interjacent there between as shown.

In the configuration illustrated, the ultrasound oscillator 60 is arranged in the front-end portion of the pad attachment portion 41, a tubular amplifying member 61 is arranged so as to surround the face of the periphery projection of the ultrasound oscillator 60, a projection portion 49 attached to the front-end side of the ultrasound oscillator 60 protrudes to the front-end side of the amplifying member 61, and a ring-shaped elastic member 49r is attached to the face of the outer circumference of this projection portion 49. The pad attachment portion 41 is inserted into the base-end side of the piston 26 in the pad portion 20 and fixed detachably to the pad portion 20 by a close contact of the ring-shaped elastic member 49r of the projection portion 49 with the face of the inner circumference of the tubular member 25. On the base-end side of the pad attachment portion 41, the face of the outer circumference of the ultrasound oscillator 60 and the face of the inner circumference of the tubular member 25 of the pad portion 20 are opposite to each other via the amplifying member 61, and both of these are not connected to each other or fixed together. A slight gap (allowance) within an amplitude Δd of the ultrasound oscillator 60 is formed between the face of the outer circumference of the ultrasound oscillator 60 and the face of the inner circumference of the amplifying member 61. Accordingly, the ultrasound oscillator 60 collides by vibration with the face of the inner circumference of the amplifying member 61 and this impact causes the amplifying member 61 to collide with the face of the inner circumference of the tubular member of the pad portion 20 with an amplitude larger than that of the oscillator 60, thereby amplifying the vibration and transmitting it to the pad portion 20. Thus, the pad portion 20 is allowed to vibrate by the amplified amplitude.

An amplitude of the ultrasound oscillator 60 is preferably determined according to the size of the abrasive particles contained in the peeling agent and the frequency. It is preferable for the amplitude to be 10 μm to 50 μm when the average particle diameter of the abrasive particles is smaller than 1000 nm and the frequency is as high as 1 kHz to 5 MHz. This amplitude is preferably amplified by the amplifying means described above. In this case, the amplitude of the abrasive pad 21 is preferably designed to be 100 μm to 500 μm.

In the peeling device of the present third embodiment configured as describe above, the frequency can be set to be of the order of MHz and peeling by this device can be achieved with significantly higher efficiency than that with the device of the second embodiment. Further, when used in the delivery method of a substance of the present invention, the probability of reaching of the delivery substance to the absorption pores is enhanced and the absorption of the delivery substance is significantly improved, both of which are achieved by activation of the tissues around the skin surface caused by the high-speed and fine vibration, high-speed and fine vibration of the delivery substance, and high-speed, minute expanding/shrinking and fine movement of the absorption pores.

Furthermore, by controlling the vibration in each axial direction using the ultrasound oscillator 60 of the orthogonal biaxial type, it is possible for rotary vibration capable of accurate scrubbing to be generated more speedily and finely, thereby allowing an improvement in peeling efficiency to a significant degree. Moreover, when such rotary vibration is used in the delivery method of a substance of the present invention described above, it is possible to achieve an enhancement in delivery efficiency to a more significant degree.

### EXAMPLE

Silica particles having particle diameters in the range of 5 nm to 50 μm were mixed with an anesthetic to prepare a peeling agent in a cream form. This peeling agent was placed in the peeling device (ultrasound vibration type) of the third embodiment described above and then the skin of a test subject was subjected to peeling for 5 minutes. After that, a needle was inserted into the peeled site and the time for pain disappearance was measured.

Further, using the same peeling agent, additional two tests, i.e. peeling for 5 minutes by hand-massage and coating only the peeling agent over the skin without providing any abrasive force such as massage or the like were carried out.

As the result, in a case where the peeling device of the ultrasound vibration type was used, an effect of the anesthesia began to appear only 5 minutes after peeling and the anesthesia was perfectly effective after about 10 minutes. Contrary to that, in a case where hand-massage was carried out, the anesthesia was perfectly effective after about 20 minutes. On the other hand, in a case where only the peeling agent was applied, the anesthesia started to be effective in about 30 minutes.

### ADVANTAGES OF THE INVENTION

As described above, various advantages that can be offered by the present invention include: peeling can be carried out without accompanying any pain, at a low cost and with ease, the peeling of a barrier on the skin surface can be carried out uniformly and accurately, and so on.

What is clamed is:

1. A delivery method of a substance via skin absorption, comprising peeling a skin surface using abrasive particles having an average particle diameter smaller than 1000 nm, applying a delivery substance to the skin surface and delivering the delivery substance from the peeled skin surface into the skin via skin absorption.

2. A delivery method of a substance via skin absorption according to claim 1, wherein the peeling is carried out by applying vibration of an amplitude of 100 μm to 1000 μm and a frequency of 1 kHz to 10 MHz to the abrasive particles, the delivery substance and the skin surface, and the delivery is carried out at the same time when the peeling is carried out in a state where the abrasive particles and the delivery substance are applied to the skin surface.

3. A delivery method of a substance via skin absorption according to claim 1 or 2, wherein globular silica particles are used as the abrasive particles.

4. A delivery method of a substance via skin absorption according to any one of claims 1 to 3, wherein abrasive particles having an antibacterial property are used as the abrasive particles.

5. A delivery method of a substance via skin absorption according to any one of claims 1 to 4, wherein abrasive particles having an average particle diameter smaller than 100 nm are used as the abrasive particles.

6. A delivery method of a substance via skin absorption according to any one of claims 1 to 5, wherein a medical substance and/or a cosmetic substance is used as the delivery substance.

7. A delivery method of a substance via skin absorption according to any one of claims 1 to 6, wherein an antibacterial agent, a disinfectant and/or a germicide are applied to the skin surface besides the abrasive particles and the delivery substance.

8. A peeling agent for delivering a substance via skin absorption, comprising abrasive particles having an average particle diameter smaller than 1000 nm and a delivery substance delivered into the skin via skin absorption, both being contained in a dispersed media.

9. A peeling agent for delivering a substance via skin absorption according to claim 8, wherein the abrasive particles are globular silica particles.

10. A peeling agent for delivering a substance via skin absorption according to claim 8 or 9, wherein the abrasive particles have an antibacterial property.

11. A peeling agent for delivering a substance via skin absorption according to any one of claims 8 to 10, wherein the abrasive particles have an average particle diameter smaller than 100 nm.

12. A peeling agent for delivering a substance via skin absorption according to any one of claims 8 to 11, wherein the delivery substance contains a medical substance and/or a cosmetic substance.

13. A peeling agent for delivering a substance via skin absorption according to any one of claims 8 to 12, wherein an antibacterial agent, a disinfectant and/or a germicide are contained besides the abrasive particles and the delivery substance.

14. A peeling device, comprising a grip portion and a pad portion attached to the grip portion, the pad portion having an abrasive pad, a storage for storing a peeling agent containing at least abrasive particles and a supply path for supplying the peeling agent stored in the storage to the abrasive pad.

15. A peeling device according to claim 14 or 15, wherein the grip portion having a pad attachment portion connected through an elastic connecting member, the pad attachment portion having a means for generating vibration, and the pad portion being attached to the pad attachment portion are configured.

16. A peeling device according to claim 15, wherein the abrasive pad has a flat front-end face and the means for generating vibration generates vibration in a direction substantially parallel to the front-end face of the abrasive pad.

17. A peeling device according to claim 16, wherein the vibration is rotary vibration.

18. A peeling device according to any one of claims 15 to 17, wherein an ultrasound oscillator is provided as the means for generating vibration, vibration generated by the ultrasound oscillator is designed to be transmitted to the abrasive pad, and an amplification means is provided for amplifying the vibration in the process of transmission of the vibration.

19. A peeling device according to any one of claims 15 to 18, wherein the peeling agent contains abrasive particles having an average particle diameter smaller than 1000 nm, and the abrasive pad are designed to be vibrated with an amplitude of 100 μm to 1000 μm and a frequency of 1 kHz to 10 MHz.

20. A peeling device according to any one of claims 14 to 19, wherein the pad portion is configured to have a front end and a base end, a tubular member provided with a partition wall in the middle thereof in the longitudinal direction, and a piston arranged on the base-end side of the partition wall within the tubular member,
wherein, the abrasive pad is configured to have a front-end portion and a base-end portion, where the base-end portion is retained on the front-end side of the partition wall within the tubular member and the front-end portion protrudes from the front end of the tubular member,
wherein, a part between the partition wall and the piston is configured to serve as the storage,
wherein, the supply path is configured to pass through the partition wall and the abrasive pad and has an opening on the outer face of the front-end portion of the abrasive pad, and
wherein, the grip portion is configured to have a front-end portion and a base-end portion, the front-end portion of the grip portion is configured to be inserted into the base-end side of the partition wall within the tubular member, and the peeling agent within the storage is designed to be supplied to the outer face of the front-end side of the abrasive pad through the supply path by inserting the piston with the front-end portion of the grip portion.

21. A peeling device according to any one of claims 14 to 19, wherein the pad portion is detachably attached to the grip portion in a way that the pad portion is disposable.

22. A disposable pad for a peeling device, comprising:
a tubular member having a front end and a base end, and a partition wall which is provided in the middle thereof in the longitudinal direction,
a piston arranged on the base-end side of the partition wall within the tubular member,
an abrasive pad having a front-end portion and a base-end portion, the base-end portion being retained on the front-end side of the partition wall within the tubular member, and the front-end portion protruding from the front end of the tubular member,
a storage for a peeling agent formed as a space between the partition wall and the piston,
a peeling agent charged in the storage and containing at least abrasive particles,
a supply path for the peeling agent extending from the storage for the peeling agent through the partition wall and the abrasive pad to an opening on the outer face of the front-end portion of the abrasive pad, and
a grip portion being configured to be inserted removably on the base-end side of the partition wall within the tubular member.
